(19) United States
(12) Patent Application Publication
(10) Pub. No.: US 2002/0166560 A1
(43) Pub. Date: Nov. 14, 2002

(54) Feminine urinary incontinence inhibitor interlabial device

(76) Inventor: Ruth A. Wolak, Taylor, MI (US)

Correspondence Address: Leonard D. McMahon, 18925 Mvirland St., Detroit, MI, 48221, US

(21) Appl. No.: 10/141,333

(22) Filed: May 8, 2002

Related U.S. Application Data
(60) Provisional application No. 60/290,278, filed on May 11, 2001.

(51) Int. Cl. A61F 5/48

(52) U.S. Cl. 128/885

(57) ABSTRACT

A feminine hygienic Urinary Incontinence Inhibitor Interlabial Device for insertion into the interlabial space; the device having a conical (or polygonal) tapered configuration which (1) facilitates vertical placement of the device into the interlabial space, and (2) by placing the conical (or polygonal) top (larger portion) of the device interlabially directly over and upon the urethra opening, thereby simulating and/or stimulating urethral external sphincter muscle function, has the utility to inhibit involuntary micturition.

### CROSS REFERENCE TO RELATED APPLICATIONS

Not Applicable

### BACKGROUND OF THE INVENTION

This invention relates to female bladder control, and more particularly to a device that, for the first time, is anatomically configured to be worn interlabially by female wearers over and upon the urethra opening to provide incontinence prevention by acting as a urethral external sphincter muscle function simulator and/or stimulus.

All manner and variety of heretofore related articles purposed for the absorption, shield, displacement and/or retention of body fluids such as urine, menses, vaginal secretions, post-coital leakage, or the like, are well known. However, none of these articles are claimed to inhibit involuntary micturition (incontinence).

### SUMMARY OF THE INVENTION

Therefore, this invention relates to a new feminine hygienic urinary incontinence inhibitor interlabial device that offers its incontinent users resultant relief and freedom through its unique method of controlling involuntary incontinence by placement of the device over the urethra opening to simulate and/or stimulate urethral external sphincter muscle function.

### DESCRIPTION OF THE DRAWING

Enclosed is a perspective drawing of a cone-shaped feminine personal hygiene product of the character described. The device can be made of a mildly porous, semi-absorbent, but firm, material.

For example, the material 10 can be any such form of cellulose, cotton, cloth, fiber, gel, etc. that is non-allergenic, and biodegradable, and that can be shaped into a semi-firm form (e.g., cone or polygon) to be vertically placed under the labia and directly on top of the urethra opening.

The urethra is a small tube leading from the floor of the bladder to the exterior opening for urinary discharge. In the female, it lies upward of the vagina, towards the anterior part of the body.

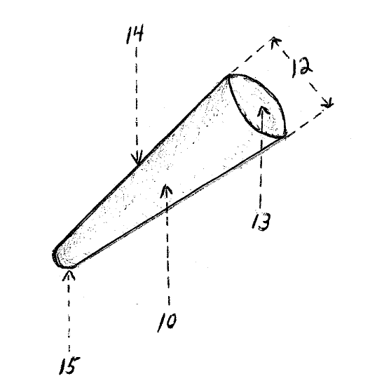

In the female, the urethra lies anterior to the vagina and extends up, in, and back for a distance of about one (1) to one and one-half (1½) inches. Accordingly, FIG. 1 represents the invention material 10 shaped as a cone which has a length (or altitude) 11 of two (2) inches, a width (or diameter) 12 of one (1) inch at its circular top 13, and curved sides 14 that slant equally into a vertex (bottom) 15.

The device, therefore, is wide at its conical (or polygonal) top 13 and narrow at its tapered bottom 15.

The device, therefore, is larger and thicker at its conical (or polygonal) top than at its narrower bottom.

The user's body (labia) holds the device securely in place.

The device would be made in various sizes to allow effectiveness and comfort for various sized users respectively.

In operation, one uses the device by placing it vertical to the vagina with the circular top 13 wide end on top of the urethra opening directly under the labia.

Therefore, when thus placed, the circular top 13 wide end of the device is anterior to the pelvic area while the narrow vertex 15 end is posterior in the direction of the vaginal canal.

The fact that the device does not have to be “inserted” into the vaginal canal, but simply “placed” in the labia and directly on top of the urethra opening, eliminates the possibility of the documented problems associated with absorbent-type devices requiring vaginal insertion.

### DETAILED DESCRIPTION OF THE INVENTION

Accordingly, an object of this invention is to provide, for the first time, feminine hygienic devices configured for the interlabial space over and upon the urethra opening to inhibit uncontrolled urine; wherein the devices have selected geometrical configurations which greatly facilitate the insertion of the top/thicker device over and upon the urethra opening; are securely retained in the interlabial space; accommodate themselves to interlabial spaces of varying sizes; and maintain their retention in the interlabial space.

Therefore, it is hereby disclosed to provide hygienic, comfortable devices for anatomical use by females to inhibit uncontrolled urinary discharge.

Said mildly absorptive urinary incontinence inhibiting devices are reasonably efficient in terms of ease of insertion and proper disposition in the interlabial space, over and upon the urethra opening.

Another object of this invention is to provide, for the first time, a feminine hygienic device having geometric conical (or polygonal) cross sections distinguished by posterior leading tapered portions of reduced transverse thickness and anterior portions of larger transverse thickness; the anterior portion greatly facilitating the insertion of the device into the interlabial space over and upon the urethra and the posterior portion having the property of laying comfortably just within the labia and preventing urinary incontinence.

A further object of this invention is to provide, for the first time, a device of the character described, wherein the placement of the anterior and posterior portions thereof provide to substantially disincline the female urinary/bladder urge and/or stress mechanism by applying pressure on the urethra to simulate and/or stimulate urethral external sphincter muscle function.

Other objects of this invention will in part be obvious and in part hereinafter pointed out.

What is claimed is:

1. A feminine hygienic, semi-absorbent, elongated device vertically placeable in its entirety into the interlabial space to inhibit uncontrolled urinary discharge by simulation and/or stimulation of the urethral external sphincter muscle function; said device can be made of a mildly porous, semi-absorbent, but firm, material of tapered length, width and thickness, and shaped into a conical (or polygonal) form with a vertical centerline; said device to be placed vertically under the vaginal labia with the larger (apex/top) portion of the device to be placed anteriorly and directly on top of the urethra opening; the smaller (vertex/bottom) portion of the device would then be extended posteriorly, and vertically, into the interlabial space; wherein said length (or altitude) of said device is greater than about 1½ inches and less than about 2½ inches, said width at the top (apex) of said device is between about 1 inch and about 1½ inches; said width and length of said device being tapered in length, width and thickness proceeding from top (apex) to bottom (vertex); to-wit, providing a shape substantially contiguous with the walls of said interlabial space and maintained thereby by the labia securely over and upon the urethra opening.

2. A urinary incontinence inhibitor device of claim 1, wherein said tapered length of semi-absorbent device is between about 1½ inches and 2½ inches.

3. A urinary incontinence inhibitor device of claim 1, wherein said tapered width of semi-absorbent device is between about 1 inches and 1½ inches.

4. A urinary incontinence inhibitor device of claim 1, wherein said tapered thickness of semi-absorbent device is tapered in thickness from top (apex) to bottom (vertex).

5. A urinary incontinence inhibitor device of claim 1, providing a shape substantially contiguous with the walls of said interlabial space and maintained by the labia thereby within said space directly over and upon the urethra opening.

6. A urinary incontinence inhibitor device of claim 1, wherein said device comprises mildly porous, semi-absorbent, but firm, material.

7. A urinary incontinence inhibitor device of claim 1, wherein said device has a vertical centerline.

8. A urinary incontinence inhibitor device as in claim 1, wherein said anterior (apex) portion is vertically extended and tapered toward said posterior (vertex) portion.

9. A urinary incontinence inhibitor device as in claim 1, wherein said top (larger) portion is vertically extended and tapered toward said bottom (smaller) portion.

10. A urinary incontinence inhibitor device as in claim 1, wherein said anterior (apex) portion has surface/face portions that are conically (or polygonally) shaped.

11. A urinary incontinence inhibitor device as in claim 1, wherein said posterior portion has tapered surface portions and surfaces.

12. A urinary incontinence inhibitor device as in claim 1, wherein said device provides for a variation of sizes to accommodate different sized users.

13. A urinary incontinence inhibitor device as in claim 1, wherein the shape and thickness of said device allows it to be maintained by the labia within the walls of the interlabial space when the anterior (apex) end is vertically placed over and directly upon the urethra opening, and the posterior (vertex) end is concomitantly placed vertically within the posterior section of the vaginal labia.

14. A urinary incontinence inhibitor device as in claim 1, wherein said device inhibits urinary incontinence in females when properly placed in the interlabial space.

15. A urinary incontinence inhibitor device as in claim 1, which by simulation and/or stimulation of the urethral external sphincter muscle function, inhibits urinary incontinence in females.
